SEQUENCE LISTING (10 sequences)

SEQ ID NO: 1 - protein, 158 aa, Human papillomavirus type 16
MHQKRTAMFQDPQERPRKLPQLCTELQTTIHEIILECVYCKQQLLRREVYDFAFRDLCIVYRDGNPYAVCDKCLKFYSKISEYRHYCYSLYGTTLEQQYNKPLCDLLIRCINCQKPLCPEEKQRHLDKKQRFHNIRGRWTGRCMSCCRSSRTRRETQL

SEQ ID NO: 2 - protein, 98 aa, Human papillomavirus type 16
MHGDTPTLHEYMLDLQPETTDLYCYEQLSDSSEEEDEIDGPAGQAEPDRAHYNIVTFCCKCDSTLRLCVQSTHVDIRTLEDLLMGTLGIVCPICSQKP

SEQ ID NO: 3 - DNA, 21 bp, Artificial Sequence
  Primer
atgcacccaaagagaactgca

SEQ ID NO: 4 - DNA, 22 bp, Artificial Sequence
  primer
attacagctgggtttctctacg

SEQ ID NO: 5 - DNA, 25 bp, Artificial Sequence
  primer
atgcatggagatacacctacattgc

SEQ ID NO: 6 - DNA, 30 bp, Artificial Sequence
  primer
ttatggtttctgagaacagatggggcacac

SEQ ID NO: 7 - DNA, 20 bp, Artificial Sequence
  primer
ttttgtagcgccagcggcca

SEQ ID NO: 8 - DNA, 20 bp, Artificial Sequence
  primer
tgcacacacccatgtgcagt

SEQ ID NO: 9 - DNA, 20 bp, Artificial Sequence
  primer
aatgtttcaggacccacagg

SEQ ID NO: 10 - DNA, 20 bp, Artificial Sequence
  primer
catacagcatatggattccc

(19) United States
(12) Patent Application Publication
(10) Pub. No.: US 2015/0044667 A1
(43) Pub. Date: Feb. 12, 2015

(54) HUMAN PAPILLOMAVIRUS 16 (HPV16) - RELATED EPILEPSY

(71) Applicant: The Trustees of the University of Pennsylvania, Philadelphia, PA (US)

(72) Inventor: Peter B. Crino, Moorestown, NJ (US)

(21) Appl. No.: 14/386,366

(22) PCT Filed: Mar. 15, 2013
(86) PCT No.: PCT/US13/32154

§ 371 Date: Sep. 19, 2014

Related U.S. Application Data
(60) Provisional application No. 61/613,115, filed on Mar. 20, 2012.

(51) Int. Cl. G01N 33/569

(52) U.S. Cl. CPC G01N 33/56983; G01N 2800/50
USPC 435/5

(57) ABSTRACT

Human papillomavirus 16 (HPV16) is identified as a cause of epilepsy. This application is directed to HPV16-related epilepsy, methods of detection and prenatal prevention, and immunogenic, therapeutic and prophylactic compositions for same. The methods of predicting or detecting human papillomavirus (HPV16)-associated epilepsy may comprise contacting a biological sample obtained from a human subject, said human subject having experienced one or more seizures, with a diagnostic reagent that can detect HPV16 or an antigen thereof, or a humoral or cell-mediated response to HPV16 or an antigen thereof, in said biological sample, wherein the presence of HPV16 or an antigen thereof or a humoral or cell-mediated response to HPV16 or an antigen thereof, is indicative of HPV16-associated epilepsy. Also provided are parallel methods of diagnosing a structural brain defect in a subject having one or more seizures.

### INCORPORATION-BY REFERENCE OF MATERIAL SUBMITTED IN ELECTRONIC FORM

Applicant hereby incorporates by reference the Sequence Listing material filed in electronic form herewith. This file is labeled “UPN_Y6170PCT_ST25”, was created on 15 Mar. 2013, and is 3.95 KB (4,049 bytes) in size.

### STATEMENT REGARDING FEDERALLY SPONSORED RESEARCH OR DEVELOPMENT

This invention was made with government support under grant no. NS045021 awarded by the National Institutes of Health. The government has certain rights in the invention.

### BACKGROUND OF THE INVENTION

Epilepsy is presently characterized by at least two unprovoked seizures. It is currently estimated to affect 50 million people worldwide with 200,000 new cases diagnosed ever year in the United States alone. Epilepsy is frequently associated with developmental malformations of the human brain, known as focal cortical dysplasia (FCD). One particular subtype of focal cortical dysplasia, type IIB (FCDIIB), is believed to form during embryonic brain development (between weeks 8 and 20 of human gestation). In a further embodiment, a malformation, or a sporadic fetal brain malformation such as a dysplasia, is detected between 20 and 28 weeks gestation, or at 24 weeks gestation. Detection may be by magnetic resonance imaging (MRI). FCDIIB is characterized by focal brain regions exhibiting disorganized laminar architecture and enlarged, dysmorphic cells known as “balloon cells” (BCs). BCs exhibit constitutive activation of the mammalian target of rapamycin complex 1 (mTORC1) signaling pathway—a known regulator of cell growth—as evidenced by enhanced phosphorylation of the ribosomal S6 protein (phospho-S6). Although aberrant mTORC1 activation can be caused by gene mutations in upstream mTORC1 effectors, the etiology of mTORC1 activation in BCs is unknown.

High-risk human papillomavirus type 16 (HPV16) is the most common cause of cervical cancer in women and has been linked to a growing number of cases of oropharyngeal cancer. The transplacental HPV infection rate among women with either genital warts, low-grade, or high-grade cervical intraepithelial lesions has been reported as 12.2%. The prevalence of HPV infection is estimated at 26%, and in many women, HPV16 infection is asymptomatic. Further, HPV16 in spermatozoa can lead to infection of ova post-fertilization. Interestingly, the cytopathic effect of HPV 16 infection on cervical epithelium results in the formation of balloon cells (also called koilocytes). Recent research showed that the E6 oncoprotein encoded by HPV16 activates mTORC1 signaling at distinct steps: by binding to TSC2 and targeting it for ubiquitin-mediated degradation, and by activating PDK1 and Akt2. In both mechanisms, mTORC1 activation is evidenced by enhanced phosphorylation of the ribosomal S6 protein (phospho-S6).

### SUMMARY OF THE INVENTION

Provided herein are methods of predicting or detecting human papillomavirus 16 (HPV16)-associated epilepsy comprising contacting a biological sample obtained from a human subject, said human subject having experienced one or more seizures, with a diagnostic reagent that can detect HPV16 or an antigen thereof, or a humoral or cell-mediated response to HPV16 or an antigen thereof, in said biological sample, wherein the presence of HPV 16 or an antigen thereof or a humoral or cell-mediated response to HPV16 or an antigen thereof, is indicative of HPV16-associated epilepsy. Also provided are parallel methods of diagnosing a structural brain defect in a subject having one or more seizures.

Further provided are methods for predicting the development of epilepsy in a human subject, comprising contacting a prenatal biological sample of the gestational mother of said human subject with a diagnostic reagent that can detect HPV16 or an antigen thereof, or a humoral or cell-mediated response to HPV16 or an antigen thereof. Additionally, methods of preventing in utero transmission of HPV16 to a human subject, or eliciting immunity therein, are provided, comprising administering immunogenic compositions, recombinant vectors, therapeutic agents or small molecules. Methods of detection of mTORC1 activation in FCDIIB as a marker for HPV16 cellular effect are also provided. Further provided is antibody detection of HPV16 E6 or E7 in brain tissue for diagnosis of FCDIIB.

Administration includes non-functional recombinant HPV16 E6 and E7 proteins (i.e., comprises a mutation ablating function, a functional mutation), antibodies to HPV16 E6 and E7, TSC2 or vectors expressing same, agents that bind HPV16 E6 and E7, agents that downregulate PDK1 and/or Akt expression in brain tissue including RNA binding same, and small molecules that block mTORC1 activation and inhibit activation of p70S6 kinase. Also provided are compositions useful in preventing epilepsy by reducing HPV16 expression or transmission.

### DETAILED DESCRIPTION OF THE INVENTION

Localized early infection with HPV16 causes disruption of normal cortical development, likely mediated by hyperactive mTORC1 signaling caused by E6 expression. FCDIIB based on localized central nervous system HPV16 infection during brain development accounts for many of the known features of FCDIIB including sporadic occurrence, enhanced mTORC1 signaling, and altered brain cytoarchitecture.

The present application provides methods of predicting and diagnosing the development of epilepsy in a human subject, including adults and pediatric subjects. The presence of seizures and HPV16 infection are indicative of disrupted cortical development and the formation of balloon cells. Further, untreated HPV16 infection during embryonic brain development (between weeks 8 and 20 of human gestation) leads to FCDIIB.

Also provided are methods of predicting of detecting HPV 16 infection by maternal prenatal or preconception screening. Further provided are methods of preventing in utero transmission of HPV 16, including reducing maternal viral load and in binding or suppressing HPV16, its proteins, or its effects. Compositions include expression vectors, antisense nucleic acids, small molecule effectors, and existing HPV16 immunogenic compositions or vaccines.

As used herein, the term “antibody,” refers to an immunoglobulin molecule which is able to specifically bind to a specific epitope on an antigen. Antibodies can be intact immunoglobulins derived from natural sources or from recombinant sources and can be immunoreactive portions of intact immunoglobulins. The antibodies useful in the present invention may exist in a variety of forms including, for example, polyclonal antibodies, monoclonal antibodies, intracellular antibodies (“intrabodies”), diabodies, Fv, Fab and F(ab)₂, as well as single chain antibodies (scFv), camelid antibodies and humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

As used herein, the term “biological sample” is intended to include any sample of biological material from a subject, in particular a human subject, including but not limited to whole blood, blood plasma, blood serum, cerebral spinal fluid (CSF), amniotic fluid, maternal peripheral blood, cervical cells, cervical secretions, vaginal fluid, placental tissue, and brain tissue.

The terms “balloon cell” or “BC” refer to enlarged, dysmorphic cells, in focal brain regions.

“DAB” refers to 3-3′-diaminobenzidine.

“FCD” refers to focal cortical dysplasia.

“FCDIIB” refers to focal cortical dysplasia type IIB.

The term “gestational mother” as used herein refers to a female human that is carrying or will carry in her womb a human fetus or embryo.

The term “human subject” as used herein includes an adult, adolescent, child, infant, baby, fetus, or embryo. The term encompasses a gestational mother unless otherwise indicated.

“LCR” refers to long control region.

As used herein, the term “pharmaceutically acceptable carrier”, “excipient”, or “vehicle” refers to a medium which does not interfere with the effectiveness or activity of an active ingredient and which is not toxic to the hosts to which it is administered. A carrier, excipient, or vehicle includes diluents, binders, adhesives, lubricants, disintegrates, bulking agents, wetting or emulsifying agents, pH buffering agents, and miscellaneous materials such as absorbants that may be needed in order to prepare a particular composition. The use of such media and agents for an active substance is well known in the art.

The terms “prevent” or “preventing” mean causing an infection, disease or disorder to not occur in a human subject. In the embodiments of the application, the terms “prevent” or “preventing” may also be used to indicate that the infection or disease is managed such that it is inactive, non-detectable, minor, or having no negative physiological effect such as those for which these embodiments are aimed to prevent or treat.

As used herein, the terms “treat” or “treating” refer to reversing, alleviating, ablating, or inhibiting the progress of a disease, or one or more symptoms of such disease, to which such term applies. Depending on the condition of the subject, the term also refers to preventing a disease, and includes preventing the onset, or preventing the symptoms associated with a disease. A treatment may be either performed in an acute or chronic way. The term also refers to reducing the severity of a disease or symptoms associated with such disease prior to affliction with the disease. Such prevention or reduction of the severity of a disease prior to affliction refers to administration of a compound or composition described herein to a subject that is not at the time of administration afflicted with the disease. “Preventing” also refers to preventing the recurrence of a disease, or of one or more symptoms associated with such disease. The terms “treatment” and “therapeutically” refer to the act of treating, as “treating” is defined above.

The amino acid sequence of HPV16 E6 is identified as SEQ ID NO: 1. The amino acid sequence of HPV16 E7 is identified as SEQ ID NO: 2.

Provided herein is a method of predicting or detecting human papillomavirus 16 (HPV16)-associated epilepsy comprising contacting a biological sample obtained from a human subject, said human subject having experienced one or more seizures, with a diagnostic reagent that can detect HPV16 or an antigen thereof, or a humoral or cell-mediated response to HPV16 or an antigen thereof, in said biological sample, wherein the presence of HPV 16 or an antigen thereof or a humoral or cell-mediated response to HPV16 or an antigen thereof, is indicative of HPV16-associated epilepsy.

Also provided is a method of diagnosing a structural brain defect, such as balloon cells or FCDIIB, in a human subject having experienced one of more seizures comprising contacting a biological sample obtained from said human subject with a diagnostic reagent that can detect human papillomavirus 16 (HPV16) or an antigen thereof, or a humoral or cell-mediated response to HPV16 or an antigen thereof, in said biological sample, wherein the presence of HPV16 or an antigen thereof or a humoral or cell-mediated response to HPV16 or an antigen thereof indicates a structural brain defect in said human subject. In another embodiment, the structural brain defect comprises ganglioglioma. In further embodiments, the diagnostic reagent can detect antibodies to HPV16 or an antigen thereof.

Further provided is a screening method for predicting the development of epilepsy in a human subject, comprising contacting a prenatal (or preconception) biological sample of the gestational mother of said human subject with a diagnostic reagent that can detect human papillomavirus 16 (HPV16) or an antigen thereof, or a humoral or cell-mediated response to HPV16 or an antigen thereof, in said biological sample, wherein the presence of HPV16 or an antigen thereof or a humoral or cell-mediated response to HPV16 or an antigen thereof is associated with an increased risk of developing epilepsy.

In one embodiment, the diagnostic methods comprise taking a biological sample likely to contain antibodies to epitopes of the E6 and E7 proteins of HPV16. This sample is preferably easy to obtain and may be serum or plasma derived from a venous or other blood sample. However, cervical secretions, cervical tissue, tissue from other body parts, or other bodily fluids are known to contain antibodies and may be used as a source of the sample. Once the peptide antigen and sample antibody are permitted to react in a suitable medium, an assay is then performed to determine to presence of an antibody-peptide reaction. Sample collection and immunoassay preparation techniques, as well as other diagnostic methods, are known to those of skill in the art and are not a limitation of the invention. For example, see U.S. Pat. No. 7,267,961 for a method of detection of HPV16 E7 antibodies. In another embodiment, the HPV E6, E7 mRNA assay and methods of U.S. Pat. No. 7,888,032 are utilized.

Further provided are methods of detection of mTORC1 activation in FCDIIB as a marker for HPV16 cellular effect. mTORC1 activation may be detected by antibodies binding any members of the mTORC1 activation cascade, including but not limited to, p70-S6 Kinase 1 (p70S6K1 or p70S6 kinase) and 4E-BP1 (eukaryotic initiation factor 4E (eIF4E) binding protein 1).

In addition, detection of HPV 16 E6 or E7 in brain tissue provides a method of diagnosing FCDIIB, among other brain defects described herein, via immunohistochemistry and Western blot analysis. These methods include obtain brain tissue sections or samples. Suitable antibodies may be prepared by conventional techniques or obtained commercially. These methods may be performed as indicated in the Examples herein, or otherwise consistent with the knowledge of one of skill in the art.

Methods and compositions of the invention are also useful for the diagnosis, treatment, and prevention of autism, neurodevelopmental disorders, and cognitive disabilities.

Embodiments of the methods and compositions described herein relating to the preventing or treatment of HPV 16 infection and of FCDIIB and other structural brain defects will be understood to include brain-specific or central-nervous-system specific delivery. Compositions or active agents thereof will be understood to require passage through the blood-brain or blood-CSF barrier via mode of delivery and formulation thereof. Delivery to a human (including fetal) brain includes any known means in the literature, including via focused ultrasound, hydrophobic molecules or small constructs, carrier-mediated transporters, in conjunction with efflux transporter blocking, osmotically, in conjunction with vasoactive molecules, via receptor-mediated transcytosis or via convention-enhanced distribution, or via nanoparticle delivery methods. Still other agents known in the art increasing passage across the blood-brain or blood-CSF barrier are known in the literature and are not a limitation of the invention. Delivery may also be made by mechanical means known to one of skill in the art, including but not limited to intracerebral implantation.

Also provided are methods of preventing in utero transmission of human papillomavirus 16 (HPV16) to a human subject comprising administering an immunogenic composition, recombinant vector, therapeutic agent, small molecule, or other agent identified herein to the gestational mother of the human subject, wherein the gestational mother is infected with HPV 16. The method may also include determining infection of the gestational mother by contacting a biological sample of the gestational mother with a diagnostic reagent that can detect human papillomavirus 16 (HPV16) or an antigen thereof, or a humoral or cell-mediated response to HPV16 or an antigen thereof, in said biological sample.

In a further embodiment, the methods comprise monitoring the viral load of HPV16 in one or more biological fluid samples of the gestational mother in adjusting treatment with the immunogenic composition, recombinant vector, therapeutic agent, small molecule, or the like.

The immunogenic composition may comprise an immunoprotective amount of a recombinant human papillomavirus 16 (HPV16) E6 protein, the E6 protein comprising a functional mutation, and a pharmaceutically acceptable carrier. The immunogenic composition may also comprise an immunoprotective amount of a recombinant human papillomavirus 16 (HPV16) E7 protein, the E7 protein comprising a functional mutation, and a pharmaceutically acceptable carrier. Further, the E6 and E7 proteins may be administered in combination. Immunogenic compositions are further described herein.

Further provided is a method of eliciting immunity in a human subject, said method comprising administering an immunogenic composition to said human subject in utero, said immunogenic composition comprising a recombinant vector that transforms cells of said human subject, wherein said recombinant vector comprises a nucleic acid molecule encoding a recombinant human papillomavirus 16 (HPV16) E6 protein, the E6 protein comprising a functional mutation, operably linked to control elements capable of effecting the expression of said nucleic acid molecule in vivo, whereby said nucleic acid molecule is expressed by the transformed cells at a level sufficient to induce immunity to HPV16 E6 in said human subject. A method of eliciting immunity in a human subject is also provided, said method comprising administering an immunogenic composition to said human subject in utero, said immunogenic composition comprising a recombinant vector that transforms cells of said human subject, wherein said recombinant vector comprises a nucleic acid molecule encoding a recombinant human papillomavirus 16 (HPV 16) E7 protein, the E7 protein comprising a functional mutation, operably linked to control elements capable of effecting the expression of said nucleic acid molecule in vivo, whereby said nucleic acid molecule is expressed by the transformed cells at a level sufficient to induce immunity to HPV 16 E6 in said human subject.

The in utero administration may be made by direct injection, by formulation of the composition for transplacental delivery, or by other means known to the skilled physician. Transplacental delivery may be accomplished via the use of oligonucleotides, recombinant vectors, and other modes both identified herein and known in the art. A recombinant vector may non-viral or viral. In one embodiment, immunity obtained via the method is

mucosal immunity.

In further embodiments, anti-HPV16 E6 and/or E7 antibodies may be prepared as described herein and directly administered to a human subject by the methods described herein.

Also provided is a method of inducing in vivo the production of anti-human papillomavirus 16 E6 antibodies comprising administering to a subject an effective antibody-inducing amount of recombinant human papillomavirus 16 (HPV 16) E6 protein, the E6 protein comprising a functional mutation, and a pharmaceutically acceptable carrier. Similarly, a method of inducing in vivo the production of anti-human papillomavirus 16 E7 antibodies is provided comprising administering to a subject an effective antibody-inducing amount of recombinant human papillomavirus 16 (HPV 16) E7 protein, the E7 protein comprising a functional mutation, and a pharmaceutically acceptable carrier.

Also provided are methods of preventing epilepsy, comprising inhibiting or treating human papillomavirus 16 infection in a human subject comprising administering to said human subject a therapeutic reagent that up-regulates the expression of TSC2 in brain tissue.

A method of preventing epilepsy is also provided comprising inhibiting or treating human papillomavirus 16 (HPV16) infection in a human subject comprising administering to said human subject TSC2 or a vector that expresses TSC2.

Further, a method of preventing epilepsy is provided comprising inhibiting or treating human papillomavirus 16 (HPV16) infection in a human subject comprising administering to said human subject a therapeutic reagent that binds HPV16 E6 protein in brain tissue. Similarly, provided is a method of preventing epilepsy comprising inhibiting or treating human papillomavirus 16 (HPV16) infection in a human subject comprising administering to said human subject a therapeutic reagent that binds HPV16 E7 protein in brain tissue.

Methods of preventing epilepsy are also provided comprising inhibiting or treating human papillomavirus 16 infection in a human subject comprising administering to said human subject a therapeutic reagent that down-regulates the expression of PDK1 and/or Akt in brain tissue. The therapeutic agent may be selected from any described herein or known to be suitable in the art, including a short nucleic acid molecule selected from the group consisting of a short hairpin RNA (shRNA), a short interfering RNA (siRNA), a double stranded RNA (dsRNA), a micro RNA, and an interfering DNA (DNAi) molecule.

Further provided are methods of preventing epilepsy comprising inhibiting or treating human papillomavirus 16 (HPV16) infection in a human subject comprising administering to said human subject a small molecule that binds HPV16 E6 protein. Also provided are methods of preventing epilepsy comprising inhibiting or treating human papillomavirus 16 (HPV16) infection in a human subject comprising administering to said human subject a small molecule that binds HPV16 E7 protein. Methods of preventing epilepsy are also provided comprising delivering to a human subject a small molecule that disrupts mTOR—substrate binding. Small molecules useful in the methods are described herein. Additional small molecules identified in the literature as binding or otherwise inhibiting proteins upregulated in HPV 16 infection and also contemplated.

The HPV16 E6 oncoprotein activates mTORC1 signaling. mTOR Complex 1 (mTORC1) is composed of mTOR, regulatory-associated protein of mTOR, mammalian LST8/G-protein β-subunit like protein (mLST8/GβL), PRAS40, and DEPTOR. Targets of mTORC1 include p70-S6 Kinase 1 (p70S6K1 or p70S6 kinase) and 4E-BP1 (eukaryotic initiation factor 4E (eIF4E) binding protein 1). HPV16 E6 expression causes an increase in mTORC1 activity through enhanced phosphorylation of mTOR and activation of downstream signaling pathways S6K and eukaryotic initiation factor binding protein 1 (4E-BP1). A decrease in TSC2 levels may also occur in HPV16 E6-expressing cells. HPV16 E6 expression causes AKT activation through the upstream kinases PDK1 and mTORC2.

In one embodiment, a method of preventing is provided, comprising inhibiting or treating human papillomavirus 16 (HPV16) infection in a human subject comprising administering to said human subject TSC2 or a vector that expresses TSC2. In another embodiment, a method of preventing epilepsy is provided comprising inhibiting or treating human papillomavirus 16 infection in a human subject comprising administering to said human subject a therapeutic reagent that up-regulates the expression of TSC2 in brain tissue. Still other methods comprise administering antisense nucleic acids, therapeutic agents and small molecules capable of binding mTOR, mTORC1, mTOR2, PDK1, Aid, p70S6K1, S6K, and 4E-BP1.

In embodiments of the invention, an effective antibody-inducing amount of a recombinant HPV 16 E6 protein is administered to a human subject in a preventative or therapeutic immunogenic composition, wherein the E6 protein comprises a functional mutation, i.e., a mutation that ablates function. In other embodiments, an effective antibody-inducing amount of a recombinant HPV 16 E7 protein is administered to a human subject in a preventative or therapeutic immunogenic composition, wherein the E7 protein comprises a functional mutation. Native HPV16 E6 and E7 amino acid sequences are provided in FIGS. 1 and 2, and may be rendered non-functional by one of ordinary skill in the art.

Techniques for generation of nucleic acid sequences and expression vectors comprising same are known and are not considered to be a limitation of the present embodiments. Mutations may be made by one or more point mutations (e.g., F47R, L50G), insertion, or deletion. Preferred E6 mutation is made in the binding site of E6 to mTOR, mTORC1, and proteins modulated through mTOR pathways.

Expression vectors for TSC2, functionally mutated E6 or E7 nucleic acids, etc., and compositions comprising same, may be prepared for delivery to a human subject according to conventional techniques. As used herein, a vector may include any genetic element including, without limitation, naked DNA, a phage, transposon, cosmid, episome, plasmid, bacteria, or a virus. As used herein, the term vector refers to a genetic element which expresses, or causes to be expressed, the desired construct that inhibits the expression of a desired functional or non-functional protein as described herein in the target cell ex vivo or in vivo.

Plasmid based systems, of which many are commercially available, may be used. In another embodiment, non-replicating recombinant viral vectors are used. Thus, in one embodiment, the vector is a non-pathogenic virus. In another embodiment, the vector is a non-replicating virus. In one embodiment, a desirable viral vector may be a retroviral vector, such as a lentiviral vector. In another embodiment, a desirable vector is an adenoviral vector. In still another embodiment, a suitable vector is an adeno-associated viral vector. Adeno, adeno-associated and lentiviruses are generally preferred because they infect actively dividing as well as resting and differentiated cells such as the stem cells, macrophages and neurons. A variety of adenovirus, lentivirus and AAV strains are available from the American Type Culture Collection, Manassas, Va., or available by request from a variety of commercial and institutional sources. Further, the sequences of many such strains are available from a variety of databases including, e.g., PubMed and GenBank.

In one embodiment, a lentiviral vector is used. Among useful vectors are the equine infectious anemia virus and feline as well as bovine immunodeficiency virus, and HIV-based vectors. A variety of useful lentivirus vectors, as well as the methods and manipulations for generating such vectors for use in transducing cells and expressing heterologous genes. In another embodiment, the vector used herein is an adenovirus vector. Such vectors can be constructed using adenovirus DNA of one or more of any of the known adenovirus serotypes. See, e.g., T. Shenk et al., Adenoviridae: The Viruses and their Replication”, Ch. 67, in FIELD'S VIROLOGY, 6^{th} Ed., edited by B. N Fields et al, (Lippincott Raven Publishers, Philadelphia, 1996), p. 111-2112; U.S. Pat. No. 6,083,716, which describes the genome of two chimpanzee adenoviruses; U.S. Pat. No. 7,247,472; WO 2005/1071093, etc. One of skill in the art can readily construct a suitable adenovirus vector to carry and express a nucleotide sequence as described herein by resort to well-known publications and patents directed to such viral vectors.

In another embodiment, the vector used herein is an adeno-associated virus vector. In another embodiment, the vector used herein is an adeno-associated virus (AAV) vector. Such vectors can be constructed using AAV DNA of one or more of the known AAV serotypes. See, e.g., U.S. Pat. No. 7,906,111 (Wilson); Gao et al, Novel Adeno-Associated Viruses From Rhesus Monkeys as Vectors for Human Gene Therapy, PNAS, vol. 99, No. 18, pp. 11854-11859, (Sep. 3, 2002); Rutledge et al, Infectious Clones and Vectors Derived from Adeno-Associated Virus (AAV) Serotypes Other Than AAV Type 2, Journal of Virology, vol. 72, pp. 309-319, (Jan. 1998). One of skill in the art can readily construct a suitable AAV vector to carry and express a nucleotide sequence as described herein.

In yet another embodiment, the vector used herein is a bacterial vector. In one embodiment, the bacterial vector is Listeria monocytogenes. Listeria monocytogenes is a food borne pathogen which has been found to be useful as a vaccine vehicle, especially in attenuated form. In one embodiment, the bacterial vector is live-attenuated or photochemically inactivated. The heterologous gene of interest can be expressed recombinantly by the bacteria, e.g., via a plasmid introduced into the bacteria, or integrated into the bacterial genome, i.e., via homologous recombination.

Generally, each of these vectors also comprises a minigene. By “minigene” is meant the combination of a selected nucleotide sequence and the operably linked regulatory elements necessary to drive translation, transcription and/or expression of the gene product in the host cell in vivo or in vitro. As used herein, “operably linked” sequences include both expression control sequences that are contiguous with the gene of interest and expression control sequences that act in trans or at a distance to control the gene of interest.

These vectors also include conventional control elements that permits transcription, translation and/or expression of the nucleic acid sequence in a cell transfected with the plasmid vector or infected with the viral vector. A great number of expression control sequences, including promoters which are native, constitutive, inducible and/or tissue-specific, are known in the art and may be utilized. In one embodiment, the promoter is an RNA polymerase promoter. In another embodiment, the promoter is an RNA polymerase promoter selected from U6, H1, T7, pol I, pol II and pol III promoters. In another embodiment, the promoter is a constitutive promoter. In another embodiment, the promoter is an inducible promoter. In one embodiment, the promoter is selected based on the chosen vector. In another embodiment, when the vector is lentivirus, the promoter is U6, H1, CMV IE gene, EF-1α, ubiquitin C, or phosphoglycerokinase (PGK) promoter. In another embodiment, when the vector is an AAV, the promoter is an RSV, U6, or CMV promoter. In another embodiment, when the vector is an adenovirus, the promoter is RSV, U6, CMV, or H1 promoters. In another embodiment, when the vector is Listeria monocytogenes, the promoter is a hly or actA promoter. Still other conventional expression control sequences include selectable markers or reporter genes, which may include sequences encoding geneticin, hygromicin, ampicillin or purimycin resistance, among others. Other components of the vector may include an origin of replication. Selection of these and other promoters and vector elements are conventional and many such sequences are available [see, e.g., Sambrook et al, and references cited therein].

These vectors are generated using the techniques and sequences provided herein, in conjunction with techniques known to those of skill in the art. Such techniques include conventional cloning techniques of cDNA such as those described in texts [Sambrook et al, Molecular Cloning: A Laboratory Manual, Cold Spring Harbor Press, Cold Spring Harbor, N.Y.], use of overlapping oligonucleotide sequences, polymerase chain reaction, and any suitable method which provides the desired nucleotide sequence.

In still other embodiments, epilepsy may be prevented by administration of a vaccine known for the prevention of HPV 16 infection, such as Gardasil® vaccine (Merck & Co.) or Cervarix® vaccine (GlaxoSmithKline) to the gestational mother of a human subject. In one embodiment, the gestational mother is administered the vaccine prior to conception. In another embodiment, the gestational mother is administered the vaccine following conception. In still a further embodiment, the gestational mother is administered the vaccine between weeks 8 and 20 of human gestation. Further, focal cortical dysplasia type IIB may be prevented by administration of Gardasil® vaccine or Cervarix® vaccine

Compositions comprising antibodies to HPV16 E6, HPV16 E7, and proteins that are upregulated during HPV16 infection are also contemplated by the application. Such compositions may be particularly useful in preventing or ameliorating the effects of HPV16 infection in a human embryo or fetus. Native HPV16 E6 and E7 amino acid sequences are provided in FIGS. 1 and 2, and may be used in the generation of said antibodies.

The antibodies of the present invention may be prepared using known techniques. Monoclonal antibodies are prepared using hydridoma technology as described by Kohler et al, Nature 256:495 (1975); Kohler et al, Eur. J. Immunol. 6:511 (1976); Kohler et al, Eur. J. Immunol. 6:292 (1976); Hammerling et al, in: Monoclonal Antibodies and T-Cell Hybridomas, Elsavier, N.Y., pages 563-681 (1981). Such antibodies produced by the methods of the invention are capable of protecting against, inhibiting, treating and/or ameliorating HPV infection.

The term “antibody” includes both polyclonal and monoclonal antibodies, as well as fragments thereof, such as, for example, Fv, Fab and F(ab).sub.2 fragments which are capable of binding antigen or hapten. Such fragments are typically produced by proteolytic cleavage, such as papain, to produce Fab fragments or pepsin to produce F(ab).sub.2 fragments. Alternatively, hapten-binding fragments can be produced through the application of recombinant DNA technology or through synthetic chemistry.

Both polyclonal and monoclonal antibodies may be employed in accordance with the embodiments of the application. Antibodies which are produced in humans or are “humanized” (i.e., non-immunogenic in a human) by recombinant or other technology are also useful. Humanized antibodies may be produced, for example, by placing an immunogenic portion of an antibody with a corresponding, but non-immunogenic portion, chimeric antibodies. See, for example, Robinson et al, International Patent Publication PCT/US86/02269; Akira et al, European Patent Application 184,187; Taniguchi, M. European Patent Application 171,496; Morrison et al, European Patent Application 173,494; Neuberger et al, PCT Application WO86/01533; Cabilly et al, European Patent Application 125,023; Better et al, Science 240:1041-1043 (1988); Liu et al, PNAS 84:3439-3443 (1987); Liu et al, J. Immunol. 139:3521-3526 (1987); Sun et al, PNAS 84:214-218 (1987); Nishimura et al, Cancer Research 47:999-1005 (1987); Wood et al, Nature 314:446-449 (1985); and Shaw et al, J. National Cancer Inst. 80:1553-1559 (1988). General reviews of “humanized” chimeric antibodies are provided by Morrison, S. L. Science 229:1202-1207 (1985) and by Oi et al, BioTechniques 4:214 (1986).

The antibodies, or antibody fragments, of the present invention can be utilized to detect, diagnose, prevent, and treat HPV 16 infection. In this manner, the antibodies or antibody fragments are particularly suited for use in immunoassays.

In some embodiments, compositions comprising short interfering RNA molecules are useful in inhibiting or treating HPV16 infection and epilepsy associated therewith. A short nucleic acid molecule useful in the compositions and in the methods described herein is any nucleic acid molecule capable of inhibiting or down-regulating gene expression of HPV16 E6, HPV16 E7, and proteins that are upregulated during HPV16 infection, particularly in the human brain or development thereof, as described herein. Upregulated proteins/protein complexes include PDK1, Aid, mTOR, mTORC1, mTORC2, p70S6K1, S6K, and 4E-BP1.

Typically, short interfering nucleic acid molecules are composed primarily of RNA, and include siRNA or shRNA, as defined below. A short nucleic acid molecule may, however, include nucleotides other than RNA, such as in DNAi (interfering DNA), or other modified bases. Thus, the term “RNA” as used herein means a molecule comprising at least one ribonucleotide residue and includes double stranded RNA, single stranded RNA, isolated RNA, partially purified, pure or synthetic RNA, recombinantly produced RNA, as well as altered RNA such as analogs or analogs of naturally occurring RNA. In one embodiment the short nucleic acid molecules of the present invention is also a short interfering nucleic acid (siNA), a short interfering RNA (siRNA), a double stranded RNA (dsRNA), a micro RNA, and/or a short hairpin RNA (shRNA) molecule. The short nucleic acid molecules can be unmodified or modified chemically. Nucleotides of the present invention can be chemically synthesized, expressed from a vector, or enzymatically synthesized.

In some embodiments, the short nucleic acid comprises between 18 to 60 nucleotides, or between 19 and 65 nucleotides. In another embodiment, the short nucleic acid molecule is a sequence of nucleotides between 25 and 50 nucleotides in length. In still other embodiments, the short nucleic acid molecule ranges up to 35 nucleotides, up to 45, up to 55 nucleotides in length, depending upon its structure. These sequences are designed for better stability and efficacy in knockdown (i.e., reduction) of gene expression. In one embodiment, the nucleic acid molecules described herein comprises 19-30 nucleotides complementary to a protein's encoding nucleic acid sense sequence, particularly an open reading frame thereof. In one embodiment, the nucleic acid molecules described herein comprise 19-30 nucleotides complementary to an antisense nucleic acid sequence strand. In one embodiment, the nucleic acid molecules described herein comprises 19-30 nucleotides complementary to a nucleic acid sense sequence.

In one embodiment, a useful therapeutic agent is a small interfering RNA (siRNA) or a siRNA nanoparticle. siRNAs are double stranded, typically 21-23 nucleotide small synthetic RNA that mediate sequence-specific gene silencing, i.e., RNA interference (RNAi) without evoking a damaging interferon response. siRNA molecules typically have a duplex region that is between 18 and 30 base pairs in length. siRNAs are designed to be homologous to the coding regions of mRNA for the protein to be suppressed, and suppress gene expression by mRNA degradation. The siRNA associates with a multi protein complex called the RNA-induced silencing complex (RISC), during which the “passenger” sense strand is enzymatically cleaved. The antisense “guide” strand contained in the activated RISC then guides the RISC to the corresponding mRNA because of sequence homology and the same nuclease cuts the target mRNA, resulting in specific gene silencing. The design of si/shRNA preferably avoids seed matches in the 3′UTR of cellular genes to ensure proper strand selection by RISC by engineering the termini with distinct thermodynamic stability. A single siRNA molecule gets reused for the cleavage of many target mRNA molecules. RNAi can be induced by the introduction of synthetic siRNA.

In one embodiment, a siRNA molecule of the invention comprises a double stranded RNA wherein one strand of the RNA is complimentary to the RNA of a protein identified herein for downregulation or inhibition. In another embodiment, a siRNA molecule of the invention comprises a double stranded RNA wherein one strand of the RNA comprises a portion of a sequence of RNA of a protein identified herein.

In one embodiment, siRNA without any chemical modification having high stability and specificity for the protein of interest, are useful as therapeutics alone, or in combination with other therapies.

In another embodiment, the short nucleic acid molecule is a small hairpin RNA (shRNA). An shRNA molecule useful in the methods and compositions described herein is generally defined as an oligonucleotide containing the about 18-23 nucleotide siRNA sequence followed by an ˜9-15 nucleotide loop and a reverse complement of the siRNA sequence. The loop nucleotides generally form a non-coding sequence. shRNAs can be prepared according to methods known to one of skill in the art and can be cloned in plasmids or in non-replicating recombinant viral vectors to endogenously/intracellularly express shRNA, which is subsequently processed in the cytoplasm to siRNA. The shRNA effects are longer lasting because they are continually produced within the cells and thus have an effect that lasts the duration of the cell's life.

In further embodiments, compositions comprising antisense nucleic acids are useful in inhibiting or treating HPV 16 infection and epilepsy associated therewith. The present invention employs oligomeric compounds, particularly antisense oligonucleotides, for use in modulating the function of nucleic acid molecules encoding HPV16 E6, HPV16 E7, and proteins that are upregulated during HPV16 infection, particularly in the human brain or development thereof, as described herein. Upregulated proteins/protein complexes include PDK1, Aid, mTOR, mTORC1, mTORC2, p70S6K1, S6K, and 4E-BP1. This is accomplished by providing antisense compounds which specifically hybridize with one or more nucleic acids encoding the protein.

As used herein, the terms “target nucleic acid” and “nucleic acid” encompass DNA encoding any of the proteins described herein for binding or downregulation, RNA (including pre-mRNA and mRNA) transcribed from such DNA, and also cDNA derived from such RNA. The specific hybridization of an oligomeric compound with its target nucleic acid interferes with the normal function of the nucleic acid. This modulation of function of a target nucleic acid by compounds which specifically hybridize to it is generally referred to as “antisense”. The functions of DNA to be interfered with include replication and transcription. The functions of RNA to be interfered with include all vital functions such as, for example, translocation of the RNA to the site of protein translation, translation of protein from the RNA, splicing of the RNA to yield one or more mRNA species, and catalytic activity which may be engaged in or facilitated by the RNA. The overall effect of such interference with target nucleic acid function is modulation of the expression of the protein. In the context of the present invention, “modulation” means either an increase (stimulation) or a decrease (inhibition) in the expression of a gene.

The preparation and targeting of antisense compounds is described in the literature and known to one of skill in the art. For example, see U.S. Pat. No. 6,174,870.

In certain embodiments, compositions useful in inhibiting or treating HPV16 infection and epilepsy associated therewith include mTOR inhibitors. mTOR inhibitors useful in these compositions include rapamycin and derivatives thereof, temsirolimus, everolimus, and ridaforolimus. In other embodiments, compositions include natural compounds, such as antioxidants, epigallocatechin gallate (EGCG), caffeine, curcumin, and resveratrol. Other inhibitors of mTOR are known in the art and are contemplated within these embodiments.

Specific embodiments of the aforementioned include the following. A recombinant human papillomavirus 16 (HPV16) E6 protein, the E6 protein comprising a functional mutation. A recombinant human papillomavirus 16 (HPV16) E7 protein, the E7 protein comprising a functional mutation. A vaccine comprising an immunoprotective amount of a recombinant HPV 16 protein comprising a functional mutation and a pharmaceutically acceptable carrier. An immunogenic composition comprising an immunoprotective amount of a recombinant HPV 16 protein comprising a functional mutation and a pharmaceutically acceptable carrier. A therapeutic or prophylactic composition comprising a vector expressing a construct that inhibits the expression of human papillomavirus 16 (HPV16) E6 protein, and a pharmaceutically acceptable carrier or diluent. A therapeutic or prophylactic composition comprising a vector expressing a construct that inhibits the expression of human papillomavirus 16 (HPV16) E7 protein, and a pharmaceutically acceptable carrier or diluent. One of the preceding compositions wherein the vector is non-viral. One of the preceding compositions wherein the vector is a non-pathogenic virus, such as a viral vector selected from the group of lentiviral, adenoviral or retroviral vectors. One of the preceding compositions wherein the vector co-expresses an anti-tumor T cell receptor or a chimeric anti-tumor T cell receptor. One of the preceding compositions wherein the vector comprises a

short hairpin (shRNA) sequence that suppresses the expression of the HPV16 E6 and/or E7 protein.

The invention also provides compositions, including pharmaceutical compositions, to practice the methods of the invention, the compositions comprising a pharmaceutically acceptable carrier. The precise dosage of antibodies, therapeutic agents, small molecules, vectors, proteins, nucleic acids, and the like, administered will vary depending upon any number of factors, including but not limited to, age, weight, and route of administration.

The compositions may be administered to a human as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, including but not limited to, age, weight, and route of administration. In preferred embodiments, the compositions are administered to a human subject or the gestational mother of a human subject between weeks 8 and 20 of human gestation.

The formulations of the pharmaceutical compositions described herein may be prepared by any method known or hereafter developed in the art of pharmacology. In general, such preparatory methods include the step of bringing together the antibodies, therapeutic agents, small molecules, vectors, proteins, nucleic acids, and the like, and a pharmaceutically-acceptable carrier or one or more other accessory ingredients, and then, if necessary or desirable, shaping or packaging the product into a desired single- or multi-dose unit.

For oligonucleotides and nucleic acids, preferred examples of pharmaceutically acceptable salts include but are not limited to (a) salts formed with cations such as sodium, potassium, ammonium, magnesium, calcium, polyamines such as spermine and spermidine, etc.; (b) acid addition salts formed with inorganic acids, for example hydrochloric acid, hydrobromic acid, sulfuric acid, phosphoric acid, nitric acid and the like; (c) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (d) salts formed from elemental anions such as chlorine, bromine, and iodine.

Pharmaceutical compositions that are useful in the methods of the invention may be prepared, packaged, or sold in formulations suitable for parenteral, ophthalmic, topical, pulmonary, buccal, intranasal, oral, rectal, vaginal, or another route of administration. In addition to the targeted nucleic acid and pharmaceutically-acceptable carrier, the pharmaceutical compositions may contain other ingredients known to enhance and facilitate drug administration. In another embodiment, the compositions are prepared for intravenous administration. In yet another embodiment, the compositions are prepared for intramuscular administration.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the antibodies, therapeutic agents, small molecules, vectors, proteins, nucleic acids, and the like, is generally equal to the dosage which would be administered to a human subject or a convenient fraction of such a dosage such as, for example, one-half or one-third of such a dosage. The relative amounts of the antibodies, therapeutic agents, small molecules, vectors, proteins, nucleic acids, and the like, the pharmaceutically acceptable carrier, and any additional ingredients in a pharmaceutical composition of the invention will vary, depending upon any number of factors, including but not limited to, age, weight, and route of administration.

Controlled- or sustained-release formulations of a pharmaceutical composition of the invention may be made using conventional technology.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intravenous, intraocular, intravitreal, subcutaneous, intraperitoneal, intramuscular, intrasternal injection, intratumoral, and kidney dialytic infusion techniques.

Formulations of a pharmaceutical composition suitable for parenteral administration comprise a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Remington's Pharmaceutical Sciences (1985, Genaro, ed., Mack Publishing Co., Easton, Pa.), which is incorporated herein by reference.

### EXAMPLES

The invention is now described with reference to the following examples. These examples are provided for the purpose of illustration only and the invention should in no way be construed as being limited to these examples but rather should be construed to encompass any and all variations that become evident as a result of the teaching provided herein. The specific embodiments described in the Examples are intended to be embodiments of the invention.

### Example 1

### HPV16 E6 Expression in FCDIIB Histopathology

A. Specimens

FCDIIB, FCDIB, FCDIIA, GG, mMCD, and TLE brain tissues were obtained following surgical resection (Academic Medical Center, the Netherlands; University College, London; University of Pennsylvania Medical Center, Philadelphia, Pa.). Specimens were targeted for resection based on standard pre-surgical evaluation with inpatient video-EEG monitoring and neuroimaging. Control brain tissues were obtained at post-mortem examination (National Disease Research Interchange, Philadelphia, Pa.; Developmental Tissue Bank, University of Maryland). All human tissue was obtained in accordance with approved institutional review board protocols at Academic Medical Center, University College Institute of Neurology, and the Perelman School of Medicine, University of Pennsylvania. FCDIIB specimens resected for the treatment of intractable epilepsy (n=50; 20 females, 30 males—See Table 1) were obtained at epilepsy centers in the U.S., U.K., and the Netherlands. Samples were histopathologically confirmed by nestin and vimentin immunoreactivity.

TABLE 1

| | | | Age/surg | | HPV 16 |
|---|---|---|---|---|---|
| Number | Diagnosis | M/F | yrs | Location | (+/−) |
| 1 | FCDIIB | m | 10 | frontal | (+) |
| 2 | FCDIIB | m | 14 | frontal | (+) |
| 3 | FCDIIB | f | 9 | frontal | (+) |
| 4 | FCDIIB | f | 41 | temporal | (+) |
| 5 | FCDIIB | m | 14 | frontal | (+) |
| 6 | FCDIIB | f | 13 | frontal | (+) |
| 7 | FCDIIB | f | 33 | frontal | (+) |
| 8 | FCDIIB | f | 37 | temporal | (+) |
| 9 | FCDIIB | f | 1 | frontal | (+) |
| 10 | FCDIIB | f | 12 | frontal | (+) |
| 11 | FCDIIB | m | 23 | temporal | (+) |
| 12 | FCDIIB | m | 29 | temporal | (+) |
| 13 | FCDIIB | m | 1 | frontal | (+) |
| 14 | FCDIIB | f | 7 m | temporal | (+) |
| 15 | FCDIIB | m | 1 | temporal | (+) |
| 16 | FCDIIB | m | 5 | temporal | (+) |
| 17 | FCDIIB | m | 14 | frontal | (+) |
| 18 | FCDIIB | m | 15 | temporal | (+) |
| 19 | FCDIIB | m | 21 | temporal | (+) |
| 20 | FCDIIB | f | 17 | frontal | (+) |
| 21 | FCDIIB | m | 3 | temporal | (+) |
| 22 | FCDIIB | f | 23 | frontal | (+) |
| 23 | FCDIIB | f | 12 | frontal | (+) |
| 24 | FCDIB | m | 12 | frontal | (+) |
| 25 | FCDIIB | m | 1 | frontal | (+) |
| 26 | FCDIIB | m | 26 | frontal | (+) |
| 27 | FCDIIB | m | 30 | frontal | (+) |
| 28 | FCDIIB | m | 44 | temporal | (+) |
| 29 | FCDIIB | f | 52 | frontal | (+) |
| 30 | FCDIIB | m | 59 | temporal | (+) |
| 31 | FCDIIB | f | 22 | frontal | (+) |
| 32 | FCDIIB | m | 52 | cingulate | (+) |
| 33 | FCDIIB | m | 26 | frontal | (+) |
| 34 | FCDIIB | m | 14 | temporal | (+) |
| 35 | FCDIIB | m | 23 | frontal | (+) |
| 36 | FCDIIB | f | 18 | temporal | (+) |
| 37 | FCDIIB | f | 31 | temporal | (+) |
| 38 | FCDIIB | m | 14 | frontal | (+) |
| 39 | FCDIIB | m | 35 | frontal | (+) |
| 40 | FCDIIB | m | 21 | frontal | (+) |
| 41 | FCDIIB | f | 15 | parietal | (+) |
| 42 | FCDIIB | m | 29 | temporal | (+) |
| 43 | FCDIIB | m | 5 | frontal | (+) |
| 44 | FCDIIB | f | 37 | temporal | (+) |
| 45 | FCDIIB | m | 24 | frontal | (+) |
| 46 | FCDIIB | f | 23 | temporal | (+) |
| 47 | FCDIIB | m | 17 | frontal | (+) |
| 48 | FCDIIB | f | 25 | frontal | (+) |
| 49 | FCDIIB | f | 15 | frontal | (+) |
| 50 | FCDIIB | f | 23 | frontal | (+) |

B. Antibodies and Immunohistochemistry. Formalin Fixed, Paraffin Embedded 6-10 μm Sections were Probed with Anti-HPV16 E6 (See Table 2).

TABLE 2

| | | | Host/ | | |
|---|---|---|---|---|---|
| Antibody | Source | Class | Isotype | Dilution | Epitope |
| Ab70 | Abcam | monoclonal | Mouse/ | 1:200 | HPV16/18 |
| | | | IgG | | E6 |
| MA1-46057 | Thermo | monoclonal | Mouse/ | 1:200 | HPV16/18 |
| | Scientific | | IgG | | E6 |
| NB100-2729 | Novus | monoclonal | Mouse/ | 1:200 | HPV16/18 |
| | Biologicals | | IgG | | E6 |
| sc-1583 | Santa Cruz | polyclonal | Goat/IgG | 1:200 | HPV16 E6 |

Sections were also probed with anti-phospho-S6 (Cell Signaling Technology™, #108D2), anti-p16INK4a (Abcam™ #ab54210), or anti-p53 antibodies overnight at 4° C. (TRIS buffer pH 7.4/2% fetal bovine serum). Probed sections were incubated in biotinylated secondary antibody, and visualized using avidin-biotin conjugation (Vectastain™ ABC Elite; Vector Labs, Burlingame, Calif. or Powervision™ Kit; Immunologic, Duiven, The Netherlands) with 3,3′-diaminobenzidine. For double-labeling studies, after incubation with primary antibodies, sections were incubated for 2 hours with Alexa Fluor® 568 and Alexa Fluor® 488 (anti-rabbit IgG or anti-mouse IgG; 1:200; Molecular Probes, Eugene, USA) and visualized with a laser scanning confocal microscope (Leica™ SP2; argon-ion laser; Wetzlar, Germany)

C. Results

There was robust cytoplasmic expression of HPV16 E6 in numerous BCs and large dysmorphic neurons in the subcortical white matter and cortical mantle of all 50 FCDIIB specimens detected by all E6 antibodies (20 females, 30 males). In accordance with varying degrees of pathological heterogeneity in FCDIIB, subregions of some specimens did not contain BCs; these regions did not exhibit E6 expression, whereas regions with high BC numbers showed extensive E6 expression. Similar to quantification of other established BC protein markers, cell count analyses in 10 representative FCDIIB specimens revealed that 79% of morphologically defined BCs expressed E6 (See Table 3).

TABLE 3

| FCDIIB | Total # | % BCs |
|---|---|---|
| Case | BCs | Expressing E6 |
| 1 | 110 | 75% |
| 2 | 95 | 94% |
| 3 | 115 | 86% |
| 4 | 96 | 87% |
| 5 | 100 | 70% |
| 6 | 112 | 71% |
| 7 | 90 | 65% |
| 8 | 111 | 81% |
| 9 | 112 | 77% |
| 10 | 98 | 86% |

E6 expression was highly specific for and exclusively linked to FCDIIB histopathology and was not detected in 36 control brain specimens including temporal lobe epilepsy specimens with no evidence of focal cortical dysplasia (FCD; n=9), or in five other types of epilepsy-associated brain malformations not characterized by BCs (FCDIB n=4, FCDIIA n=7, tuberous sclerosis complex (TSC) n=5, ganglioglioma (GG) n=6, temporal lobe epilepsy (TLE) n=9, and mild malformation of cortical development (MCD) n=1), as shown in Table 4 (controls, no malformation; n=4).

TABLE 4
Absence of HPV 16 E6 in Non-FCDIIB Cases

| | | | Age at | | |
|---|---|---|---|---|---|
| | | | Surgery | | HPV 16 |
| Case | Diagnosis | Gender | (years) | Specimen Location | (+/−) |
| 1 | MCD | F | 4 | Parietal | − |
| 2 | FCDIB | M | 19 | Frontal | − |
| 3 | FCDIB | M | 23 | Temporal | − |
| 4 | FCDIB | F | 16 | Frontal | − |
| 5 | FCDIB | M | 24 | Temporal | − |
| 1 | FCDIIA | M | 5 | Frontal | − |
| 2 | FCDIIA | M | 1 | Frontal | − |
| 3 | FCDIIA | M | 3 | Frontal | − |
| 4 | FCDIIA | M | 13 | Temporal | − |
| 5 | FCDIIA | F | 1 | Parietal | − |
| 6 | FCDIIA | F | 22 | Temporal | − |
| 7 | FCDIIA | M | 17 | Frontal | − |
| 1 | TSC | M | 32 | Parietal | − |
| 2 | TSC | F | 42 | Frontal | − |
| 3 | TSC | M | 16 | Temporal | − |
| 4 | TSC | M | 1 | Temporal | − |
| 5 | TSC | F | 14 | Subependymal giant | − |
| | | | | cell astrocytoma | |
| | | | | (SEGA) | |
| 1 | Control | F | 73 | Temporal | |
| 2 | Control | M | 44 | Frontal | |
| 3 | Control | F | 46 | Frontal | |
| 4 | Control | M | 59 | Frontal | |
| 1 | GG | M | 27 | Temporal | |
| 2 | GG | F | 19 | Temporal | |
| 3 | GG | M | 34 | Temporal | |
| 4 | GG | M | 31 | Frontal | |
| 5 | GG | M | 19 | Temporal | |
| 6 | GG | F | 22 | Temporal | |
| 1 | TLE | M | 36 | Temporal | |
| 2 | TLE | F | 19 | Temporal | |
| 3 | TLE | M | 35 | Temporal | |
| 4 | TLE | M | 24 | Temporal | |
| 5 | TLE | F | 29 | Temporal | |
| 6 | TLE | M | 30 | Temporal | |
| 7 | TLE | F | 31 | Temporal | |
| 8 | TLE | F | 45 | Temporal | |
| 9 | TLE | F | 43 | Temporal | |

p16INK4a, a transcription factor highly associated with HPV infection in cervical and oropharyngeal cancers was detected immunohistochemically within BCs in all FCDIIB cases analyzed (n=20), but not in surrounding brain tissue without BCs or in control brains. Additionally, because p53 degradation is promoted by E6 expression and in cervical cancers, we examined p53 expression. Contrary to previous reports, our specimens exhibited very low p53 expression in astrocytes within the lesions and no expression in BCs.

E6 antibody specificity was confirmed by Western blot and immunocytochemistry in HeLa and CaSki cervical cancer cell lines. E6 was not detected in control brain, FCDIA, in cell lines generated from normal control individuals and patients with tuberous sclerosis complex—an autosomal dominant disorder with brain histopathology similar to FCDIIB—or in U87 and U87vIII glioma lines. These findings highlight the specificity of E6 immunodetection in FCDIIB.

### Example 2

### HPV16 E6 DNA and mRNA Expression in FCDIIB

A. PCR

Template DNA was extracted in blinded fashion from 10 randomly selected FCDIIB specimens containing E6 labeled BCs and 19 control specimens. Viral cross-contamination of tissue blocks at the time of processing was unlikely since cases were sectioned in 3 different pathology labs.

DNA was extracted using QIAam DNA FFPE™ Tissue Kit (QIAGEN® #56404). PCR was performed in PCR-safe, contaminant free stations. Primers amplifying the entire HPV16 E6 (5′-ATGCACCCAAAGAGAACTGCA-3′ forward (SEQ ID NO: 3); 5′-ATTACAGCTGGGTTTCTCTACG-3′ reverse (SEQ ID NO: 4) and E7 (5′-ATGCATGGAGATACACCTACATTGC-3′ forward (SEQ ID NO: 5); 5′-TTATGGTTTCTGAGAACAGATGGGGCACAC-3′ reverse (SEQ ID NO: 6) coding regions and HPV16 LCR (5′-TTTTGTAGCGCCAGCGGCCA-3′ forward (SEQ ID NO: 7); 5′-TGCACACACCCATGTGCAGT-3′ reverse (SEQ ID NO: 8) were used to detect viral genomic DNA from FCDIIB specimens. DNA was subjected to 35 cycles of PCR amplification.

Primers were designed to detect HPV16 E6 (primers directed against HPV18 E6 did not amplify a product; not shown). A 477 bp amplicon, confirmed by sequencing to be HPV16 E6 (NCBI #U34127), was detected in all FCDIIB specimens and CaSki cells, but not in control brain specimens or in an HPV-negative cervical cancer cell line, C33A.

B. Reverse Transcriptase PCR

RNA was extracted using RNeasy™ FFPE Kit (QIAGEN #73504). Synthesis of cDNA and subsequent RT-PCR were performed using OneStep™ RT-PCR Kit (QIAGEN #210210). E6 specific RT-PCR primers amplifying E6 cDNA were used (5′-AATGTTTCAGGACCCACAGG-3′ forward (SEQ ID NO: 9); 5′-CATACAGCATATGGATTCCC-3′ reverse (SEQ ID NO: 10). RT-PCR using specific E6 primer pairs identified E6 mRNA in 5 FCDIIB cases.

C. In Situ Hybridization

Detection of HPV DNA to determine whether HPV 16 DNA is integrated or episomal was accomplished using GenPoint™ HPV Biotinylated DNA Probe (#Y1443) and GenPoint™ Tyramide Signal Amplification System for Biotinylated Probes (#K0620) from Dako. In situ hybridization was achieved as previously described (Montag, M. et al. Evaluation of two commercialized in situ hybridisation assays for detecting HPV-DNA in formalin-fixed, paraffin-embedded tissue. Arch Gynecol Obstet, 284, 999-1005 (2011)), with the exception of the use of 200 μg/ml proteinase K in water and 1:50 DAB for 5 minutes.

FCDIIB specimens (n=7) were probed with a biotinylated HPV DNA probe and visualized with DAB. A previous study using this probe demonstrated robust hybridization to HPV16 DNA in CaSki and SiHa cell lines. In all FCDIIB specimens, HPV16 DNA was found exclusively in BCs. HPV DNA was not present in regions of FCDIIB with no BCs or in control brain tissue (n=4). The diffuse distribution of DAB signal in BCs suggests that HPV16 DNA exists in an episomal conformation. As further corroboration of HPV16, the HPV16 E7 oncoprotein gene and the upstream long control region were also detected in FCDIIB cases by PCR.

D. Immunohistochemistry

Double fluorescence immunohistochemistry was performed on FCDIIB specimens using antibodies against E6 and phospho-S6 (Ser235/236), a robust biomarker for mTORC1 activation in FCDIIB. Phospho-S6 and E6 proteins were highly co-expressed in FCDIIB, particularly in BCs and dysmorphic neurons. A few cells expressing E6 did not exhibit phospho-S6, but virtually all of the phospho-S6 labeled cells expressed E6. Cortical tubers, which exhibit, TORC1 activation as a consequence of either mutations in TSC1 or TSC2 genes (but not E6 expression), did not inhibit E6 immunoreactivity. mTORC1 activation is not a feature of control brain tissue, FCDI, and MCD; this was supported by the absence of E6 in these tissues.

### Example 3

### E6 Expression and Cortical Malformation

A. In Utero Electroporation

Introduction of plasmids by in utero electroporation was achieved according to Saito, T. In vivo electroporation in the embryonic mouse central nervous system. Nat. Protoc. 1, 1552-8 (2006). A plasmid encoding E6 and an RFP reporter plasmid (5 μl each) were microinjected into the lateral ventricle of fetal mouse brains (n=3) at embryonic day 14 (E14). In control mice, an RFP-only plasmid was injected (n=5). The lateral aspects of the brain were exposed to a brief electrical field pulse (40V) by a forceps-type electrode (CUY21EDIT Square-Wave Electroporator) to effect passage of the plasmid into cells. Fetal pups were euthanized at E19 and 10 μm thick frozen brain sections were probed with E6 and phospho-S6 antibodies. Sections were visualized by fluorescence (Leica DM4000 B microscope) or confocal (Zeiss LSM510) microscopy.

B. Results

Progenitor cells born on E14 achieve a cortical laminar destination of layer II-III by E19, and brains transfected with RFP plasmid alone exhibited RFP+ cells in layer II-III at E19. Histological examination of the co-transfected brains on E19 by fluorescence microscopy revealed a focal cortical malformation where 74% of RFP+ cells—all of which were E6 immunoreactive—failed to reach their appropriate layer II-III destination and accumulated in the subcortical white matter and ventricular/subventricular (VZ/SVZ) zones. In co-transfected brains, RFP+ neurons that reached cortical layer II-III were not E6 immunoreactive. Co-labeling with phospho-S6 (Ser235/236) revealed that transfected RFP+ cells within the focal malformation in the VZ/SVZ expressed phospho-S6. These results demonstrate that E6 expression in neural progenitor cells during fetal brain development alters cell migration and cortical lamination in association with enhanced mTORC1 activation.

### SUPPORTING LITERATURE

Arbeit, J. M., Münger, K., Howley, P. M., & Hanahan, D. Neuroepithelial carcinomas in mice transgenic with human papillomavirus type 16 E6/E7 ORFs. Am. J. Pathol. 142, 1187-1197 (1993).
Banks, L. et al. Identification of human papillomavirus type 17 E6 polypeptide derived from human cervical cancers. J. gen. Virol. 68, 1351-1359 (1987).
Bishop, J. A. & Westra, W. H. Human papillomavirus-related small cell carcinoma of the oropharynx. Am. J. Surg. Pathol. 35, 1679-1684 (2011).
Blümcke, I. et al. The clinicopathologic spectrum of focal cortical dysplasias: a consensus classification proposed by an ad hoc Task Force of the ILAE Diagnostic Methods Commission. Epilepsia 52, 158-174 (2011).
Boer, K., et al Immunohistochemical characterization of the out-of frame splice variants GFAP Delta164/Deltaexon 6 in focal lesions associated with chronic epilepsy. Epilepsy research 90, 99-109 (2010).
Chamberlain, W. A. & Prayson, R. A. Focal cortical dysplasia type II (malformations of cortical development) aberrantly express apoptotic proteins. Appl Immunohistochem Mol Morphol. 5, 471-476 (2008).
Crino, P. B. mTOR: A pathogenic signaling pathway in developmental brain malformations. Trends Mol Med. 17, 734-42 (2011).
Doorbar, J. Molecular biology of human papillomavirus infection and cervical cancer. Clinical Science. 110, 525-541 (2006).
Dunne, E. F. et al. Prevalence of HPV infection among females in the United States. JAMA 297, 813-819.
Evans, M. F. Aliesky, H. A., & Cooper, K. Optimization of biotinyl-tyramide-based in situ hybridization for sensitive background-free applications on formalin-fixed, paraffin-embedded tissue specimens. BMC Clinical Pathology, 3, (2003).
Faridi, R., Zahra, A., Khan, K., & Idrees, M. Oncogenic potential of human papillomavirus (HPV) and its relation with cervical cancer. Virol J. 8, 269-276 (2011).
Foresta, C. et al. Mechanism of human papillomavirus binding to human spermatozoa and fertilizing ability of infected spermatozoa. PLoS One. 6, e15036 (2011).
Fûle, T. et al. The presence of human papillomavirus 16 in neural structures and vascular endothelial cells. Virology 348, 289-296 (2006).
Grajkowska, W. et al. Expression of tuberin and hamartin in tuberous sclerosis complex-associated sporadic cortical dysplasia of Taylor's balloon cell type. Folia Neuropathol. 46, 43-38 (2008).
Kostopoulo, E. et al. Different patterns of p16 immunoreactivity in cervical biopsies: correlation to lesion grade and HPV detection, with a review of the literature. Eur J Gynaecol Oncol. 32, 54-61 (2011).
Lamparello, P. et al. Developmental lineage of cell types in cortical dysplasia with balloon cells. Brain 130, 2267-2276 (2007).
Lu, Z. et al. Human papillomavirus 16 E6 oncoprotein interferences with insulin signaling pathway by binding to tuberin. J. Biol. Chem 279, 35664-35670 (2004).
McLeod K. Prediction of human papilloma virus antigen in cervical squamous epithelium by koilocyte nuclear morphology and “wart scores”: confirmation by immunoperoxidase. J Clin Path. 40, 323-328 (1987).
Middeldorp, J. et al. GFAPdelta in radial glia and subventricular zone progenitors in the developing human cortex. Development 137, 313-321 (2010).
Montag, M. et al. Evaluation of two commercialised in situ hybridisation assays for detecting HPV-DNA in formalin-fixed, paraffin-embedded tissue. Arch Gynecol Obstet, 284, 999-1005 (2011).
Orlova, K. A. et al. Early progenitor cell marker expression distinguishes type II from type I focal cortical dysplasias. J Neuropathol Exp Neurol. 69, 850-63 (2010).
Rombaldi, R. L., Serafini, E. P., Mandelli, J., Zimmermann, E., & Losquiavo, K. P. Transplacental transmission of Human Papillomavirus. Virol J. 5, 106-120 (2008).
Saito, T. In vivo electroporation in the embryonic mouse central nervous system. Nat. Protoc. 1, 1552-8 (2006).
Scheffner, M., Werness, B. A., Hulbregtse, J. M., Levine, A. J., & Howley, P. M. The E6 oncoprotein encoded by human papillomavirus types 16 and 18 promotes the degradation of p53. Cell 63, 1129-1136 (1990).
Schick, V. et al. Differential Pi3K-pathway Activation in Cortical Tubers and Focal Cortical Dysplasias with Balloon Cells. Brain Pathology 17, 165-173 (2007).
Spangle, J. M. & Münger, K. The human papillomavirus type 16 E6 oncoprotein activates mTORC1 signaling and increases protein synthesis. J. Virol. 84, 9398-9407 (2010).
Tota, J. E., Chevarie-Davis, M., Richardson, L. A., Devries, M., & Franco, E. L. Epidemiology and burden of HPV infection and related diseases: implications for prevention strategies. Prey Med. 53, Suppl 1:S12-21 (2011).
Zhang Y., et al. BRCA1 interaction with human papillomavirus oncoproteins. J Biol Chem. 280, 33165-77 (2005).

Any document (including but not limited to any patent, patent application, publication, and website) listed herein is hereby incorporated herein by reference in its entirety, as well as the Sequence Listing submitted herewith and the entirety of U.S. Provisional Patent Application No. 61/613,115, filed Mar. 20, 2012. In addition, the Summary of the Invention, the Examples, and the Claims of U.S. Provisional Patent Application No. 61/613,115, filed Mar. 20, 2012, are expressly incorporated by reference into the text of the present application. While these developments have been disclosed with reference to specific embodiments, it is apparent that other embodiments and variations of this invention are devised by others skilled in the art without departing from the true spirit and scope of the developments. The appended claims include such embodiments and variations thereof

TABLE 5
Sequence Listing Free Text
The following information is provided for sequences containing
free text under numeric identifier <223>.

| SEQ ID NO: | |
|---|---|
| (containing free text) | Free text under <223> |
| 1 | N/A |
| 2 | N/A |
| 3 | primer |
| 4 | primer |
| 5 | primer |
| 6 | primer |
| 7 | primer |
| 8 | primer |
| 9 | primer |
| 10 | primer |

1: A method of predicting or detecting human papillomavirus 16 (HPV16)-associated epilepsy comprising:
contacting a biological sample obtained from a human subject, said human subject having experienced one or more seizures, with a diagnostic reagent that can detect HPV16 or an antigen thereof, or a humoral or cell-mediated response to HPV16 or an antigen thereof, in said biological sample, wherein the presence of HPV16 or an antigen thereof or a humoral or cell-mediated response to HPV16 or an antigen thereof, is indicative of HPV 16-associated epilepsy.

2: A method of diagnosing a structural brain defect in a human subject having experienced one of more seizures comprising:
contacting a biological sample obtained from said human subject with a diagnostic reagent that can detect human papillomavirus 16 (HPV16) or an antigen thereof, or a humoral or cell-mediated response to HPV16 or an antigen thereof, in said biological sample,
wherein the presence of HPV 16 or an antigen thereof or a humoral or cell-mediated response to HPV 16 or an antigen thereof indicates a structural brain defect in said human subject.

3: The method according to claim 2, wherein the structural brain defect comprises balloon cells.

4: The method according to claim 4, wherein the structural brain defect is focal cortical dysplasia type IIB (FCDIIB).

5: The method according to claim 2, wherein the structural brain defect comprises gangliglioma.

6: The method according to claim 2, wherein the diagnostic reagent can detect antibodies to HPV16 or an antigen thereof.

7: A method for predicting the development of epilepsy in a human subject, comprising contacting a prenatal biological sample of the gestational mother of said human subject with a diagnostic reagent that can detect human papillomavirus 16 (HPV16) or an antigen thereof, or a humoral or cell-mediated response to HPV16 or an antigen thereof, in said biological sample,
wherein the presence of HPV 16 or an antigen thereof or a humoral or cell-mediated response to HPV16 or an antigen thereof is associated with an increased risk of developing epilepsy.

8: The method according to claim 7, wherein the biological sample is blood.

9: The method according to claim 8, wherein the biological sample is maternal peripheral blood.

10: The method according to claim 6, wherein the biological sample is cervical cells.

11: The method according to claim 6, wherein the biological sample is amniotic fluid.

12: The method according to claim 6, wherein the biological sample is placental tissue.

13-53. (canceled)

54. The method according to claim 1, wherein the diagnostic reagent can detect antibodies to HPV16 or an antigen thereof.
